(19) United States
(12) Patent Application Publication
(10) Pub. No.: US 2025/0332089 A1
(43) Pub. Date: Oct. 30, 2025

(54) WATER-SOLUBLE MEDICINE DELIVERY FORM

(71) Applicant: Heman BEKELE, Annandale, VA (US)

(72) Inventor: Heman BEKELE, Annandale, VA (US)

(21) Appl. No.: 18/649,011

(22) Filed: Apr. 29, 2024

(51) Int. Cl. A61K 9/00; A61K 9/107; A61K 9/127; A61K 31/4745; B82Y 5/00

(52) U.S. Cl. CPC A61K 9/0014; A61K 9/10; A61K 31/4745; B82Y 5/00

(57) ABSTRACT

A medicine delivery form is configured to deliver medicine to a user's skin for absorption and includes a water-soluble base and a plurality of nanoparticle clusters distributed within the water-soluble base. Each nanoparticle cluster includes a plurality of hydrophobic nanoparticles and a plurality of active agent particles. The plurality of nanoparticle clusters are configured to be absorbed into the user's skin. The water-soluble base is configured to remain on the outer surface of the user's skin. The nanoparticle clusters and the water-soluble base are configured form a lather or residue on the user's skin upon the water-soluble base being rubbed against the user's skin and water being applied to the water-soluble base. The nanoparticle clusters and the water-soluble base are configured so that when the water-soluble base is removed from the user's skin, adding water to the lather or residue removes the lather or residue from the user's skin.

### FIELD OF INVENTION

The invention relates to medicine delivery forms, and particularly to medicine delivery forms that are water-soluble and deliver medicine by way of absorption through a user's skin.

### BACKGROUND

Skin cancers are the most common form of cancer accounting for at least 40% of all diagnosed cancers worldwide. There are three main types of skin cancer: basal-cell carcinoma, squamous-cell skin carcinoma, and malignant melanoma.

Traditionally, skin cancers are treated by way of surgery or excision. In extreme cases, radiotherapy and chemotherapy are used to treat skin cancers. Such treatments can be costly and are extremely invasive. In addition, the treatments require the guidance of a doctor and are performed by a specialist.

Aside from the high cost and invasiveness of the treatment, some areas of the world lack medical resources needed to treat skin cancers (e.g., lack of qualified doctors and specialists). Without the necessary medical resources, otherwise treatable forms of cancer become fatal.

### SUMMARY

The present technology is directed to a more affordable, less invasive, and widely available treatment for skin cancers.

A first aspect of the technology relates to a medicine delivery form configured to deliver medicine to a user's skin for absorption. The medicine delivery form includes a water-soluble base; a plurality of nanoparticle clusters distributed within the water-soluble base, each nanoparticle cluster comprising a plurality of hydrophobic nanoparticles and a plurality of active agent particles, wherein the plurality of nanoparticle clusters are configured to be absorbed into the user's skin, wherein the water-soluble base is configured to remain on the outer surface of the user's skin, wherein the nanoparticle clusters and the water-soluble base are configured form a lather or residue on the user's skin upon the water-soluble base being rubbed against the user's skin and water being applied to the water-soluble base, and wherein the nanoparticle clusters and the water-soluble base are configured so that when the water-soluble base is removed from the user's skin, adding water to the lather or residue removes the lather or residue from the user's skin.

The hydrophobic nanoparticles are lipid based. The active agent particles are tricyclic organic molecules. The tricyclic organic molecule is imidazoquinolines. The water-soluble base is soap. The nanoparticle clusters are evenly distributed throughout the water-soluble base. The nanoparticle clusters are unevenly distributed within the water-soluble base. The size of the hydrophobic nanoparticles is within a range of 20 nm to 100 nm. The water-soluble base is liquid soap. Each nanoparticle cluster has a shell configuration in which the active agent particles are surrounded by the hydrophobic nanoparticles.

In yet another aspect of the technology, a medicine delivery form is configured to deliver medicine to a user's skin for absorption. The medicine delivery form includes a bar of soap; a plurality of nanoparticle clusters distributed within the bar of soap, each nanoparticle cluster comprising a plurality of hydrophobic nanoparticles and a plurality of hydrophilic nanoparticles, wherein the nanoparticle clusters are configured to be absorbed into the user's skin, wherein the bar of soap is configured to remain on the outer surface of the user's skin, wherein the nanoparticle clusters and the bar of soap are configured form a lather or residue on the user's skin upon the bar of soap being rubbed against the user's skin and water being applied to the bar of soap, and wherein the nanoparticle clusters and the bar of soap are configured so that when the water-soluble base is removed from the user's skin, adding water to the lather or residue removes the lather or residue from the user's skin.

The hydrophobic nanoparticles are lipid based. The hydrophilic nanoparticles are tricyclic organic molecules. The tricyclic organic molecule is imidazoquinolines. The nanoparticle clusters are evenly distributed throughout the bar of soap. The nanoparticle clusters are unevenly distributed within the bar of soap. The size of the hydrophobic nanoparticles is within a range of 20 nm to 100 nm. Each nanoparticle cluster has a shell configuration in which the hydrophilic nanoparticles are surrounded by the hydrophobic nanoparticles.

### BRIEF DESCRIPTION OF THE DRAWINGS

FIG. 1 illustrates a medicine delivery form in relation to a treatment site.

FIG. 2A illustrates an arrangement of nanoparticle clusters in the medicine delivery form before use.

FIG. 2B illustrates another arrangement of nanoparticle clusters in the medicine delivery form before use.

FIG. 2C illustrates another arrangement of nanoparticle clusters in the medicine delivery form before use.

FIG. 2D illustrates another arrangement of nanoparticle clusters in the medicine delivery form before use.

FIG. 3 illustrates a nanoparticle cluster and its components.

FIG. 4 illustrates another configuration of the nanoparticle cluster.

### DETAILED DESCRIPTION OF EXAMPLE NON-LIMITING EMBODIMENTS

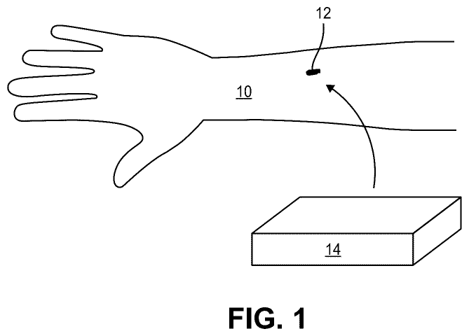

FIG. 1 shows a human arm 10 with a cancerous region 12. The cancerous region 12 may be region of cancerous cells such as basal-cell carcinoma, squamous-cell skin carcinoma, and malignant melanoma. In addition, although a human arm 10 is shown in FIG. 1, the location of the cancerous region 12 can be anyway on the human body. FIG. 1 also shows a medicine delivery form 14 that delivers medicine to the cancerous region 12 by way of absorption through the user's skin.

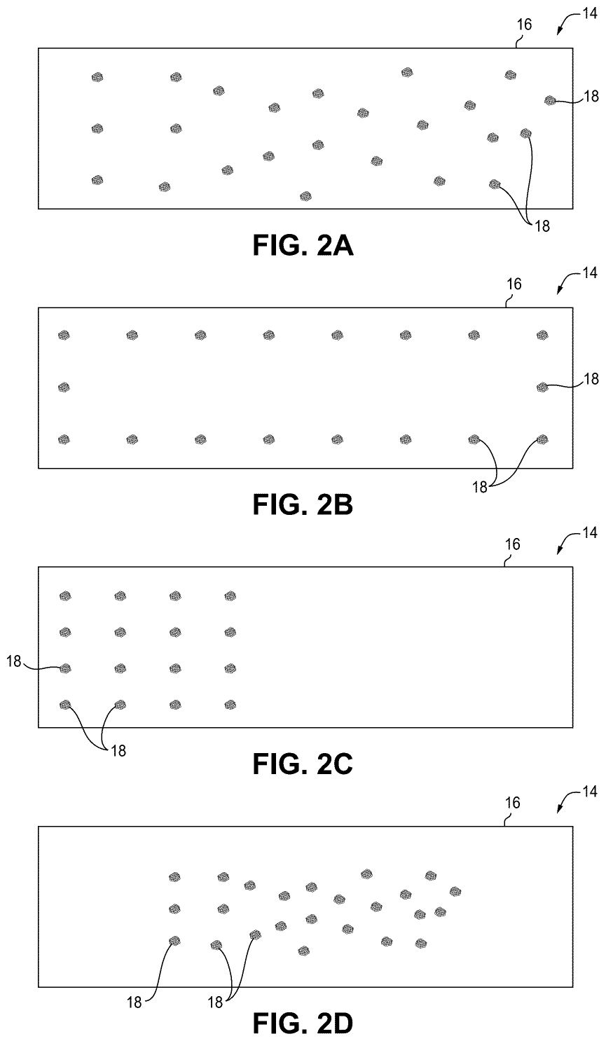

FIG. 2 illustrates a schematic representation of the medicine delivery form 14, which may include a water-soluble base 16 impregnated with nanoparticle clusters 18. The medicine delivery form 14 is designed to be applied to the user's skin so that some or all of the nanoparticle clusters 18 can be absorbed through the user's skin.

The water-soluble base 16 may be any water-soluble substance capable of being impregnated with nanoparticle clusters 18. In one configuration, the water-soluble base 16 may be in the form of fatty acid salts or soap. When in the form of soap, the water-soluble base 16 may be formed from a mixture of triglycerides and sodium hydroxide (for harder forms of soap) or a mixture of triglycerides and potassium hydroxide (for softer forms of soap). It is contemplated that the triglycerides may be produced from fats, oils, and/or free fatty acids. Some existing topical creams may be a source of the fats, oils, and/or free fatty acids. Alternatively, the fats, oils, and/or free fatty acids may be provided by natural oils, butters, natural wax, and/or other natural or artificial sources.

The nanoparticle clusters 18 are the components of the medicine delivery form 14 that penetrates the user's skin and delivers the medicine to the cancerous region 12. As discussed above, the water-soluble base 16 may be impregnated with the nanoparticle clusters 18. In other words, the water-soluble base 16 may provide a supporting structure for the nanoparticle clusters 18 until the nanoparticle clusters 18 are delivered to and/or are absorbed into the user's skin. It is contemplated that the nanoparticle structures 18 may be distributed in the water-soluble base 16 in any number of arrangements.

For example, as shown in FIG. 2A, the nanoparticle clusters 18 may be evenly distributed throughout the water-soluble base 16. In this configuration, the dosage of medicine delivered to the cancerous region 12 may be the same regardless of the side of the water-soluble base 16 being applied to the user's skin. In addition, in this configuration, the medicine may continue to be delivered at a consistent rate to the cancerous region 12 as long as the water-soluble base 16 is applied to the user's skin.

FIG. 2B shows another configuration of the medicine delivery form 14 in which the nanoparticle clusters 18 are distributed along the perimeter of the water-soluble base 16. In this arrangement, the medicine delivery form 14 will stop delivering the medicine after a predetermined percentage of the water-soluble base 16 has dissolved or worn away. This may be helpful in limiting the dosage applied to the cancerous region 12 because the water-soluble base 16 can wear away to a point where no more nanoparticle clusters 18 are available for delivery.

FIG. 2C shows a configuration in which the nanoparticle clusters 18 are distributed within a fraction of the water-soluble base 16. For example, one quarter, one third, or one half of the water-soluble base 16. It is also contemplated that the nanoparticle clusters 18 may be distributed along only one surface of the water-soluble base 16 or less than all of the surfaces of the water-soluble base 16. In this arrangement, the nanoparticle clusters 18 may be distributed to the cancerous regions 12 when the medicine delivery form 14 is applied to the user's skin in only one or a few predetermined orientations. This may allow the medicine delivery form 14 to be used in both medical and nonmedical applications depending on the orientation of the medicine delivery form 14 when applied to the user's skin. It may also allow the user to handle the medicine delivery form 14 without an unintended part of the user's body receiving the nanoparticle clusters 18 through contact with the medicine delivery form 14 (e.g., the user's hand holding the medicine delivery form 14).

FIG. 2D shows a configuration in which the nanoparticle clusters 18 are distributed toward the center of the medicine delivery form 14 and away from the perimeter of the medicine delivery form 14. In other words, the nanoparticle clusters 18 may be spaced a predetermined distance from all outer surfaces of the medicine delivery form 14. This arrangement may allow the user to handle the medicine delivery form 14 without an unintended part of the user's body receiving the nanoparticle clusters 18 through contact with the medicine delivery form 14 (e.g., the user's hand holding the medicine delivery form 14). In particular, the only surface that wears away is the one that engages the user's skin. Accordingly, that side of the medicine delivery form 14 will be the only surface through which the nanoparticle clusters 18 are delivered.

It should be understood that the distribution of the nanoparticle clusters 18 within the water-soluble base 16 is not limited to the examples provided above. In addition, the medicine delivery form 14 any combination of nanoparticle cluster distributions discussed above.

Each nanoparticle cluster 18 may include one or more hydrophobic nanoparticles 20 and an active agent 22. The hydrophobic nanoparticles 20 may be, for example, lipid based particles. It is contemplated that the hydrophobic nanoparticles 20 may have a spherical shape or any other shape capable of forming the nanoparticle cluster 18. In addition, the hydrophobic nanoparticles 20 may be between 10 and 1000 nm.

The active agent 22 may include the molecules that treat the cancerous cells in the cancerous regions 12. In other words, active agent 22 may be the actual medicine in the medicine delivery form 14. It is contemplated that the active agent 22 may include tricyclic organic molecules such as, for example, imidazoquinolines. It is also contemplated that the active agent 22 may be made of hydrophilic nanoparticles.

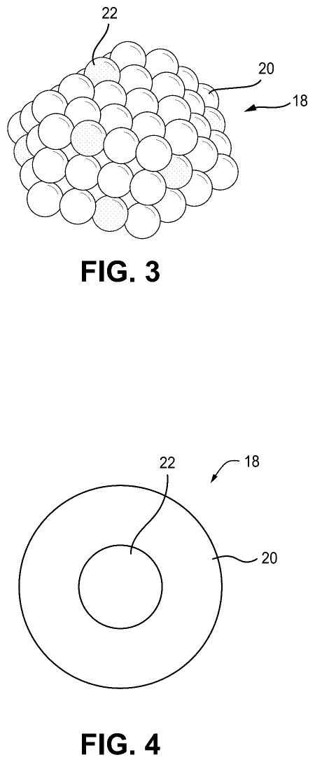

FIG. 3 illustrates an exemplary structure for the nanoparticle clusters 18 in which the active agent particles 22 are intermixed with the hydrophobic nanoparticles 20. FIG. 4 illustrates another exemplary structure for the nanoparticles clusters 18 in which the hydrophobic nanoparticles 20 surround the active agent particles 22 so that the active agent particles 22 are at the center of the nanoparticle cluster 18 and the hydrophobic nanoparticles 20 form the outer portion of the nanoparticle cluster 18. In other words, the nanoparticle cluster 18 of FIG. 3 has a shell-type configuration in which the active agent particles 22 form the core and the hydrophobic nanoparticles 20 form the shell.

The hydrophobic nanoparticles 20 may act as the carrier for the active agent particles 22 and deliver the active agent particles 22 to the targeted cancerous cells. In addition, the overall diameter of the nanoparticle cluster 18 may be within a range of 5 nm to 1 μm, within a range of 45 nm to 100 nm, e.g., 25 nm, 45 nm, 70 nm, 100 nm, or 300 nm.

In order to treat the cancerous region 12, water is applied to the medicine delivery form 14 as the medicine delivery form 14 is applied to and rubbed against the user's skin to create a lather or residue on the user's skin. The lather includes particles of the water-soluble base 16 and the nanoparticle clusters 18. At least some of the released particles (i.e, the nanoparticle clusters 18) are absorbed into the user's skin and migrate to the cancerous cells in the cancerous region 12. However, none of the particles of the water-soluble base 16 are absorbed into the user's skin. Water or other liquid may be applied to the water-soluble base 16 or the user's skin at the cancerous region 12 to wash the lather off of the user's skin. It is contemplated that the medicine delivery form 14 may be in the form of a solid bar of soap, liquid soap, or other compound that releases particles and/or forms a lather in the presence of water when in contact with the user's skin.

The medicine delivery form 14 may be applied to just the cancerous region 12 (as opposed to the entire body as is the case with conventional washing soap). In addition, the medicine delivery form 14 may be applied to the user's skin for one to ten minutes. In addition, the soap may simply rest against the user's skin for the whole or part of the duration of the treatment session, rubbed against the user's skin for the whole or part of the duration of the treatment session, a combination of both. After the preset time period has elapsed, the medicine delivery form 14 may be removed from the user's skin and the remaining lather or residue may be washed away by applying water. The duration of the treatment session may depend on the target dosage to be delivered to the cancerous region 12. It is contemplated that there may be one to three treatment sessions per day and that the treatment sessions may be repeated for weeks or months until the cancerous cells are no longer present.

While at least one exemplary embodiment of the present invention(s) is disclosed herein, it should be understood that modifications, substitutions and alternatives may be apparent to one of ordinary skill in the art and can be made without departing from the scope of this disclosure. This disclosure is intended to cover any adaptations or variations of the exemplary embodiment(s). In addition, in this disclosure, the terms “comprise” or “comprising” do not exclude other elements or steps, the terms “a” or “one” do not exclude a plural number, and the term “or” means either or both, unless this application states otherwise. Also, the terms “approximately”, “about”, and “substantially” encompass a range of plus or minus 15%. Furthermore, characteristics or steps which have been described may also be used in combination with other characteristics or steps and in any order unless the disclosure or context suggests otherwise. This disclosure hereby incorporates by reference the complete disclosure of any patent or application from which it claims benefit or priority.

1. A medicine delivery form configured to deliver medicine to a user's skin for absorption, the medicine delivery form comprising:
a water-soluble base; and
a plurality of nanoparticle clusters distributed within the water-soluble base, each nanoparticle cluster comprising a plurality of hydrophobic nanoparticles and a plurality of active agent particles,
wherein the plurality of nanoparticle clusters are configured to be absorbed into the user's skin,
wherein the water-soluble base is configured to remain on the outer surface of the user's skin,
wherein the nanoparticle clusters and the water-soluble base are configured form a lather or residue on the user's skin upon the water-soluble base being rubbed against the user's skin and water being applied to the water-soluble base,
and wherein the nanoparticle clusters and the water-soluble base are configured so that when the water-soluble base is removed from the user's skin, adding water to the lather or residue removes the lather or residue from the user's skin.

2. The medicine delivery form of claim 1, wherein the hydrophobic nanoparticles are lipid based.

3. The medicine delivery form of claim 1, wherein the active agent particles are tricyclic organic molecules.

4. The medicine delivery form of claim 3, wherein the tricyclic organic molecules are imidazoquinolines.

5. The medicine delivery form of claim 1, wherein the water-soluble base is soap.

6. The medicine delivery form of claim 1, wherein the nanoparticle clusters are evenly distributed throughout the water-soluble base.

7. The medicine delivery form of claim 1, wherein the nanoparticle clusters are unevenly distributed within the water-soluble base.

8. The medicine delivery form of claim 1, wherein the size of the hydrophobic nanoparticles is within a range of 20 nm to 100 nm.

9. The medicine delivery form of claim 1, wherein the water-soluble base is liquid soap.

10. The medicine delivery form of claim 1, wherein each nanoparticle cluster has a shell configuration in which the active agent particles are surrounded by the hydrophobic nanoparticles.

11. A medicine delivery form configured to deliver medicine to a user's skin for absorption, the medicine delivery form comprising:
a bar of soap; and
a plurality of nanoparticle clusters distributed within the bar of soap, each nanoparticle cluster comprising a plurality of hydrophobic nanoparticles and a plurality of hydrophilic nanoparticles,
wherein the nanoparticle clusters are configured to be absorbed into the user's skin,
wherein the bar of soap is configured to remain on the outer surface of the user's skin,
wherein the nanoparticle clusters and the bar of soap are configured form a lather or residue on the user's skin upon the bar of soap being rubbed against the user's skin and water being applied to the bar of soap, and
wherein the nanoparticle clusters and the bar of soap are configured so that when the soap is removed from the user's skin, adding water to the lather or residue removes the lather or residue from the user's skin.

12. The medicine delivery form of claim 11, wherein the hydrophobic nanoparticles are lipid based.

13. The medicine delivery form of claim 11, wherein the hydrophilic nanoparticles are tricyclic organic molecules.

14. The medicine delivery form of claim 13, wherein the tricyclic organic molecules are imidazoquinolines.

15. The medicine delivery form of claim 11, wherein the nanoparticle clusters are evenly distributed throughout the bar of soap.

16. The medicine delivery form of claim 11, wherein the nanoparticle clusters are unevenly distributed within the bar of soap.

17. The medicine delivery form of claim 11, wherein the size of the hydrophobic nanoparticles is within a range of 20 nm to 100 nm.

18. The medicine delivery form of claim 11, wherein each nanoparticle cluster has a shell configuration in which the hydrophilic nanoparticles are surrounded by the hydrophobic nanoparticles.
